(19) United States
(12) Patent Application Publication
(10) Pub. No.: US 2019/0086319 A1
(43) Pub. Date: Mar. 21, 2019

(54) METHODS OF EVALUATING A CELLULAR SAMPLE FOR HER-2/NEU EXPRESSION AND COMPOSITIONS FOR PRACTICING THE SAME

(71) Applicant: Becton, Dickinson and Company, MC110, NJ (US)

(72) Inventors: Eileen SNOWDEN, Cary, NC (US); Rainer BLAESIUS, Chapel Hill, NC (US); Friedrich HAHN, Durham, NC (US); Warren PORTER, Chapel Hill, NC (US); Mitchell FERGUSON, Clayton, NC (US)

(21) Appl. No.: 16/082,507

(22) PCT Filed: Mar. 7, 2017
(86) PCT No.: PCT/US17/21205

§ 371 Date: Sep. 5, 2018

Related U.S. Application Data
(60) Provisional application No. 62/306,592, filed on Mar. 10, 2016.

(51) Int. Cl. G01N 15/14; G01N 33/574; G01N 33/52; G01N 33/533; G01N 33/58; C07K 16/30

(52) U.S. Cl. CPC G01N 15/1459; G01N 33/57415; G01N 33/5748; G01N 2015/1006; G01N 33/533; G01N 33/582; C07K 16/3015; G01N 33/52

(57) ABSTRACT

Methods of evaluating a cellular sample for HER-2/neu expression are provided. Aspects of the methods include flow cytometrically obtaining fluorescence emission data from a cellular sample fluorescently labelled HER-2/neu specific binding member and employing the sample fluorescence emission data with standard fluorescence emission data to obtain a value of fluorescently labelled HER-2/neu specific binding members bound per cell. Compositions, devices and kits for performing these methods are also provided

### CROSS-REFERENCE TO RELATED APPLICATION

Pursuant to 35 U.S.C. § 119(e), this application claims priority to the filing date of U.S. Provisional Patent Application No. 62/306,592, filed Mar. 10, 2016; the disclosure of which application is herein incorporated by reference.

### INTRODUCTION

Human epidermal growth factor receptor 2 (HER-2 or HER-2/neu) is a member of the epidermal growth factor receptor (EGFR) family of receptor tyrosine kinases, which constitute a signaling network that plays a critical role in proliferation and survival of breast carcinoma cells. HER-2/neu is involved in normal growth and development of glandular tissue when expressed at normal levels, but abnormal overexpression or amplification of HER-2/neu disrupts normal cellular modulation to promote an aggressive cancer phenotype in glandular tissue. This process is mediated by oligomerization of HER-2/neu with other EGFR family members which in turn phosphorylates numerous downstream molecules and activates several signaling cascades.

Preclinical and clinical studies have revealed that HER-2/neu overexpression is an important phenomenon that occurs from the early stage of oncogenesis, and plays a critical role in cancer growth and progression. HER-2/neu is overexpressed in about 20-30% of invasive breast cancer cases. Its overexpression is indicative of a more aggressive (malignant) cancer and is associated with poor prognosis. A number of therapeutic compounds for breast cancer specifically target the HER-2/neu oncogene.

For example, Trastuzumab (Herceptin®) is a recombinant humanized monoclonal antibody that targets the extracellular domain of the HER-2/neu protein. The binding of Herceptin to the extracellular domain of HER-2/neu inhibits the heterodimerization of HER-2/neu with other HER receptor family members, which is important in HER-2-mediated signaling, and thus blocks the activation of downstream signaling cascades, resulting in decreased cell proliferation, increased apoptosis and decreased angiogenesis. Additional therapeutic compounds targeting HER-2/neu include: Lapatinib (Tykerb®); Pertuzumab (Perjeta®); and Ado-trastuzumab emtansine (Kadcyla®).

An accurate HER-2/neu overexpression determination in a breast cancer tumor sample is an important parameter in determining whether to treat a subject with a HER-2/neu-targeting therapeutic. The most commonly used techniques for detecting HER-2/neu status include (i) assessing HER-2/neu gene amplification by fluorescence in-situ hybridization (FISH), and (ii) assessing HER-2/neu protein overexpression by immunohistochemistry (IHC; an IHC 3+ score indicates a positive result; an IHC 0 or 1+ score indicates a negative result; and an IHC 2+ score is equivocal). It is recommended that IHC 2+ scores be retested with FISH for HER-2/neu gene amplification.

Both IHC and FISH have disadvantages. For example, IHC has low detection sensitivity relative to other molecular genetic techniques, and varying results depending on the sensitivity and specificity of antibodies used to detect HER-2. Also, since the representation of IHC results is highly subjective, it is prone to show inter-observer variability (e.g., between users using identical reagents). FISH has drawbacks in that it takes a relatively long amount of time, requires the use of a fluorescent probe about 10 times more expensive than antibodies used in IHC, and does not directly detect cancer cells. In addition, the FISH assay is difficult to interpret when different areas of a slide show differences in expression or when genotypic abnormalities of the reference chromosome CEP17 are present.

### SUMMARY

Methods of evaluating a cellular sample for HER-2/neu expression are provided. Aspects of the methods include flow cytometrically obtaining fluorescence emission data from a cellular sample fluorescently labelled HER-2/neu specific binding member and employing the sample fluorescence emission data with standard fluorescence emission data to obtain a value of fluorescently labelled HER-2/neu specific binding members bound per cell. Compositions, devices and kits for performing these methods are also provided.

In certain embodiments, the disclosed methods quantitatively assess and report HER-2/neu antigen expression on the surface of solid tumor cells in suspension for the purpose of subpopulation identification and quantification. In some embodiments, the methods quantitatively measure a solid tumor antigen, e.g., HER-2/neu antigen, using flow cytometry in parallel with an internal standard, for example BD QuantiBRITE™ PE stained standard beads, which is a fluorescence quantitation system to estimate antibodies bound per cell (ABC) based on a standard curve generated from flow cytometric data of the stained standard beads. The antigen, e.g., HER-2/neu, expression from a cell suspension prepared from a solid tumor is quantified and can be expressed as the number of antibody molecules bound per cell (ABC). This measurement may be further transformed into a ratio-metric readout by dividing the ABC value for the cells (or a subpopulation thereof) by the measured value for the low peak of the standard curve. The reported measurement may also be transformed to a fluorescence peak width distribution (rCV) to indicate the variability within the sample.

The automated quantitative measurement of HER-2 and the mathematical expression of the sample fluorescence provide a significant improvement over currently used assays, e.g., assays for HER-2/neu gene duplication (FISH) or gene expression (IHC). Furthermore, tumor subpopulations within the primary sample can be identified and quantitated based on antigen expression levels. The disclosed methods provide valuable identification and measurement of discreet populations of cells for tumor antigen expression measurement not possible with current methods.

### BRIEF DESCRIPTION OF THE FIGURES

The invention may be best understood from the following detailed description when read in conjunction with the accompanying drawings. Included in the drawings are the following figures:

FIG. 1 shows a standard curve correlating Log FL2 (Y-axis) with Log PE molecules bound per bead (X-axis) generated using BD QuantiBRITE™ beads. The measured fluorescence value of a stained cell suspension can be translated to an exact measured value of antibodies bound per cell (ABC) by measuring against this standard curve.

FIG. 2 shows a histogram (top) of HER-2/neu protein by flow cytometry on a cell line as well as a table (bottom) showing the quantitative antigen density (expressed as Antibodies Bound per Cell (ABC)), the ratio-metric comparison to the internal standard, and the measured robust coefficient of variation of intensity (rCV) for all cells and subpopulations 1 and 2.

FIG. 3 shows a composite histogram (left) of HER-2/neu expression on six cell populations: 3 ATCC breast cancer cell lines (1. MCF7; 2. MDA MB321, and 3. SKBR3) and 3 JAX PDX tumor models (4. BR0851; 5. BR1367; and 6. BR0291). The inset in each histogram of the composite shows the calculated ABC for each population. The table (bottom right) shows the HER-2/neu ratio-metric comparison (Ratio) and rCV for the three PDX tumor models as well as the ABC values.

FIG. 4 shows a graph demonstrating the correlation of the calculated HER-2/neu antibodies bound per cell (ABC) by flow cytometry as described herein with mRNA expression data from gene expression for 4 PDX tumor models (BR0744, BR0901, BR1367, and BR0851).

FIG. 5 shows a comparison of HER-2/neu expression for six human breast clinical tumor samples, designated Conversants 1 to 6. The histograms of HER-2/neu are shown for each, with background staining shown as a line (no fill) and the HER-2/neu stained sample shown filled in. ABC values for all cells (Total) and cells above background staining (Above FMO) are shown in the table. The conventional HER-2/neu scores are also shown in the table. Conversant samples 1-3 and 5 are negative for HER-2/neu expression by conventional methods. Conversant 4 is positive for HER-2/neu expression by IHC. Conversant 6 was not tested by IHC or FISH.

FIG. 6 shows a histogram of HER-2/neu expression by flow cytometry for Conversant 4. The ABC, Ratio and rCV for each different population indicated in the histogram (i.e., all, HER-2 High, HER-2 Intermediate, and HER-2 low) is shown in the table. The sample contains a complex mixture of populations expressing varying levels of HER-2/neu protein expression.

### DETAILED DESCRIPTION

Methods of evaluating a cellular sample for HER-2/neu expression are provided. Aspects of the methods include flow cytometrically obtaining fluorescence emission data from a cellular sample fluorescently labelled HER-2/neu specific binding member and employing the sample fluorescence emission data with standard fluorescence emission data to obtain a value of fluorescently labelled HER-2/neu specific binding members bound per cell. Compositions, devices and kits for performing these methods are also provided.

Embodiments of the invention include methods for quantitative and automated flow cytometry-based protein expression analysis of cells from solid tumors. In some instances, the methods provide a measurable output to meet the current standard for all newly diagnosed and metastatic tumor assessment based on HER-2/neu expression. In addition to providing a quantitative readout for HER-2/neu expression in the form of antibodies bound per cell (ABC), the output can be expressed as a ratio to provide comparison between samples (e.g., samples run at different times and with different reagents) and also by using coefficient of variation (rCV) to express tumor cell heterogeneity and quantitatively identify sample subpopulations. Methods of the invention provide valuable identification and measurement of discreet populations of cells for HER-2/neu measurement not possible with current methods. The methods disclosed herein are an improvement over solid tumor analytical methods currently in use in the clinic. Compositions, devices and kits for performing these methods are also provided.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

The terms “HER-2” or “HER-2/neu” are used interchangeably to refer to human epidermal growth factor receptor 2. HER-2/neu is encoded by the ERBB2 gene and is a member of the human epidermal growth factor receptor (HER/EGFR/ERBB) family. Other names for HER-2/neu include: receptor tyrosine-protein kinase erbB-2, CD340 (cluster of differentiation 340), proto-oncogene Neu, and ERBB2. The canonical amino acid sequence of HER-2/neu has been given UniProt accession number P04626-1.

The term “cellular sample” as used herein includes any biological specimen obtained from a patient or subject that contains cells. Cellular samples include, without limitation, cells from whole blood (e.g., peripheral blood mononuclear cells), ductal lavage fluid, lymph (e.g., disseminated tumor cells of the lymph node), bone marrow aspirate, fine needle aspirate (e.g., harvested by random periareolar fine needle aspiration), a tissue sample (e.g., tumor tissue) such as a biopsy of a tumor (e.g., needle biopsy) or a lymph node (e.g., sentinel lymph node biopsy), and the like. In certain embodiments, the cellular sample is from a solid tumor of the breast, e.g., a breast cancer biopsy. Cellular samples can be in the form of a cell suspension (or a “single cell suspension” as referred to in the art), e.g., a sample containing dissociated cells derived from an initial solid tissue specimen, e.g., tumor tissue.

A “biopsy” refers to the process of removing a tissue sample for diagnostic or prognostic evaluation, and to the tissue specimen itself. Any biopsy technique known in the art can be applied to the methods and compositions of the present invention. The biopsy technique applied will generally depend on the tissue type to be evaluated and the size and type of the tumor (i.e., solid or suspended (i.e., blood or ascites)), among other factors. Representative biopsy techniques include excisional biopsy, incisional biopsy, needle biopsy (e.g., core needle biopsy, fine-needle aspiration biopsy, etc.), surgical biopsy, and bone marrow biopsy.

The term “subject” or “patient” in the context of the disclosed methods refers to an individual from whom a cellular sample or biopsy is derived and analyzed. Subjects include humans, but can also include other animals such as, e.g., other primates, rodents, canines, felines, equines, ovines, porcines, and the like.

The term “binding member” as used herein refers to any agent (e.g., a protein, small molecule, and the like) that specifically binds to a marker of a target cell, e.g., HER-2/neu. The terms “specific binding”, “specifically binds”, and the like, refer to the preferential binding to a molecule relative to other molecules or moieties in a solution/reaction mixture (including on a cell surface). With respect to a target protein, “specifically binds” refers to the preferential association of an antibody or other binding member, in whole or part, with a specific polypeptide. In some embodiments, the affinity between binding member and the marker of the rare target cell to which it specifically binds when they are specifically bound to each other in a binding complex is characterized by a K_{d} (dissociation constant) of 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, including 10⁻¹⁵ M or less. “Affinity” refers to the strength of binding, increased binding affinity being correlated with a lower K_{d}.

In certain aspects, the binding members may be antibodies, or antigen-binding fragments thereof. As used herein, the term “antibodies” includes polyclonal or monoclonal antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be further conjugated to other moieties, such as members of specific binding pairs, e.g., biotin (member of biotin-avidin specific binding pair), or otherwise labeled with a detectable label, such as an enzyme, hapten, or fluorophore (or fluorescent label), and the like. Also encompassed by the term are Fab′, Fv, F(ab′)2, and or other antibody fragments that retain specific binding to antigen, and monoclonal antibodies.

By “fluorophore” or “fluorescent label” is meant any label that absorbs light at a first wavelength (or range of wavelengths) and emits light at a second, different wavelength (or range of wavelengths). In general, the emitted light from a fluorescent labels has a longer wavelength, and therefore lower energy, than the absorbed wavelength. Examples of fluorescent labels include, but are not limited to, phycobiliproteins (e.g., phycoerythrin (PE), phycocyanin (PC), allophycocyanin (APC)), rhodamine, fluorescein, alexa fluor, cascade blue, tetramethylrhodamine, Texas red, and the like, as well as those listed in greater detail below.

### Methods

As summarized above, aspects of the present disclosure provide methods of evaluating a cellular sample for oncogene expression, e.g., HER-2/neu expression, by flow cytometry. This evaluated expression can be employed to diagnose a subject for a disease, e.g., cancer, to determine a therapeutic regimen, to determine a prognosis, and/or to evaluate the efficacy of treatment.

The initial cellular sample for evaluation is obtained from any desired subject or host. In some instances, the host or subject is a subject having, or suspected of having, cancer, e.g., breast cancer. As such, the initial cellular sample can include cells from a tissue biopsy, e.g., a tumor biopsy. In certain embodiments, the tumor biopsy is a breast cancer biopsy. Obtaining an initial cell sample can include receiving the cell sample from a third party or harvesting the cell sample directly from the subject. For flow cytometric analysis, the cell sample needs to be in the form of a cell suspension. Thus, in certain embodiments, the initial cell sample obtained is in the form of a cell suspension. For example, a user can obtain a cell suspension from a third party for evaluation. In other embodiments, the initial cell sample obtained is not in the form of a cell suspension, e.g., in the form of a solid tissue or cellular aggregate, and thus the user produces a cell suspension from such obtained initial cell sample. Any convenient method for generating a cell suspension from tissue or cellular aggregate, including a solid tumor/biopsy tissue, may be employed. Such methods can include mechanical, chemical, and/or enzymatic dissociation treatments in any desired combination. Thus, in certain embodiments, the initial cell sample is subjected to tissue disruption processing steps to generate a cell suspension.

The cellular sample obtained may, in certain embodiments, be a labelled cellular sample that is already in a form amenable for flow cytometric analysis. In such embodiments, the cellular sample has been contacted with a directly or indirectly fluorescently labelled oncogene expression product-specific binding member. By directly labelled is meant that the binding member is covalently bound to a detectable fluorescent label, where by indirectly labeled is meant that the binding member and fluorescent label are bound non-covalently, e.g., through the binding of a first member of a binding pair and a second member of a binding pair, where the first member of the binding pair is attached to the oncogene expression product-specific binding member (e.g., an antibody) and the second member of the binding pair is attached to the fluorescent label. Examples of binding pairs include biotin and streptavidin, digoxigenin and anti-digoxigenin antibody, and the like. In certain embodiments, the oncogene expression product-specific binding member is one that specifically binds to the HER-2/neu protein, e.g., a HER-2/neu specific labelled antibody or binding fragment thereof. In other embodiments, the initial cellular sample is in the form of a cell suspension that is not yet labelled for oncogene expression detection. In such embodiments, the labelled cellular sample is produced by contacting the initial cellular sample with a fluorescently labelled oncogene expression product-specific binding member (e.g., a fluorescently labelled HER-2/neu specific binding member). In certain embodiments, the fluorescently labelled oncogene expression product-specific binding member includes a fluorescently labelled antibody, where in certain embodiments, the fluorescently labelled antibody is specific for HER-2/neu. Any convenient HER-2/neu specific binding member may be employed.

Any convenient fluorescent label that is detectable by flow cytometry can be employed. In certain embodiments, the fluorescent labels have an absorbance maximum ranging from 300 to 700, such as from 350 to 650 and including from 400 to 600 nm. In some instances, the emission maximum of the labels ranges from about 400 to 800, such as from 425 to 775 and including from 450 to 750 nm. Examples of fluorescent labels include, but are not limited to: 4-acetamido-4′-isothiocyanatostilbene-2,2′disulfonic acid; acridine and derivatives such as acridine, acridine orange, acridine yellow, acridine red, and acridine isothiocyanate; 5-(2′-aminoethyl)aminonaphthalene-1-sulfonic acid (EDANS); 4-amino-N-[3-vinylsulfonyl) phenyl]naphthalimide-3,5 disulfonate (Lucifer Yellow VS); N-(4-anilino-1-naphthyl)maleimide; anthranilamide; Brilliant Yellow; coumarin and derivatives such as coumarin, 7-amino-4-methylcoumarin (AMC, Coumarin 120), 7-amino-4-trifluoromethylcouluarin (Coumaran 151); cyanine and derivatives such as cyanosine, Cy3, Cy5, Cy5.5, and Cy7; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylaminocoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein isothiocyanate (FITC), fluorescein chlorotriazinyl, naphthofluorescein, and QFITC (XRITC); fluorescamine; IR144; IR1446; Green Fluorescent Protein (GFP); Reef Coral Fluorescent Protein (RCFP); Lissamine™; Lissamine rhodamine, Lucifer yellow; Malachite Green isothiocyanate; 4-methylumbelliferone; ortho cresolphthalein; nitrotyrosine; pararosaniline; Nile Red; Oregon Green; Phenol Red; B-phycoerythrin; o-phthaldialdehyde; pyrene and derivatives such as pyrene, pyrene butyrate and succinimidyl 1-pyrene butyrate; Reactive Red 4 (Cibacron™ Brilliant Red 3B-A); rhodamine and derivatives such as 6-carboxy-X-rhodamine (ROX), 6-carboxyrhodamine (R6G), 4,7-dichlororhodamine lissamine, rhodamine B sulfonyl chloride, rhodamine (Rhod), rhodamine B, rhodamine 123, rhodamine X isothiocyanate, sulforhodamine B, sulforhodamine 101, sulfonyl chloride derivative of sulforhodamine 101 (Texas Red), N,N,N′,N′-tetramethyl-6-carboxyrhodamine (TAMRA), tetramethyl rhodamine, and tetramethyl rhodamine isothiocyanate (TRITC); riboflavin; rosolic acid and terbium chelate derivatives; xanthene or combinations thereof, among other fluorophores. In certain embodiments, the fluorophore is a fluorescent dye such as rhodamine, coumarin, cyanine, xanthene, polymethine, pyrene, dipyrromethene borondifluoride, napthalimide, phycobiliprotein, peridinium chlorophyll proteins, conjugates thereof or a combination thereof. In some instances, the fluoresence label is a phycobiliprotein, (e.g., phycoerythrin (PE), phycocyanin (PC), allophycocyanin (APC)), rhodamine, fluorescein, alexa fluor, cascade blue, tetramethylrhodamine, Texas red, and the like.

Once a labelled cellular sample is obtained, the method includes (i) flow cytometrically assaying the labelled cellular sample to obtain sample fluorescence emission data from the fluorescently labelled oncogene expression product-specific binding member, and (ii) employing the sample fluorescence emission data and standard fluorescence emission data obtained from a set of standard fluorescently labelled beads to obtain a value of fluorescently labelled oncogene expression product-specific binding members bound per cell in the cell sample. In some instances, the standard fluorescently labelled beads are labelled with the same fluorescent label as the fluorescently labelled oncogene expression product-specific binding member (e.g., they are both labeled with phycoerythrin (PE)). In this way, the cellular sample is evaluated for the level of oncogene expression. As noted above, in certain embodiments the oncogene is HER-2/neu, and thus the expression product-specific binding member is a HER-2/neu specific binding member. In certain of these embodiments, the fluorescently labelled HER-2/neu specific binding member is a fluorescently labelled HER-2/neu specific antibody.

The set of standard fluorescently labelled beads employed in the disclosed methods may include two or more distinct populations of beads, each having a different known amount of a conjugated fluorescent label, e.g., a “low amount” and a “high amount”. The set of standard fluorescently labelled beads may include two or more, three or more, four or more, five or more, six or more, seven or more, eight or more, nine or more, ten or more, etc., distinct populations of beads, each having a different known amount of a conjugated fluorescent label. In some embodiments, the standard fluorescently labelled beads employed in the methods described herein includes from two to six distinct populations of beads, e.g., four distinct populations of beads. Any convenient standard beads with two or more distinct populations can be employed, e.g., QuantiBRITE™ standard beads (BD Biosciences, San Jose Calif.).

In certain embodiments, the method includes obtaining the standard fluorescence emission data for the standard fluorescently labelled beads. In certain cases, the standard fluorescence emission and cell sample fluorescence emission data are obtained at the same time. In such embodiments, the standard fluorescently labelled beads and the labelled cellular sample are present in the same sample container and analyzed for fluorescence emission using the same excitation/emission wavelength determination. Thus, in some embodiments, the method includes contacting an initial cellular sample with a fluorescently labelled oncogene expression product-specific binding member (e.g., a fluorescently labeled anti-HER-2/neu antibody) and a set of standard fluorescently labelled beads labelled with the same fluorescent label as the fluorescently labelled oncogene expression product-specific binding member to obtain the labelled cellular sample. Such contacting can include introducing the initial cellular sample into a container in that contains (i) a set of standard fluorescently labelled beads and (ii) the fluorescently labelled oncogene expression product-specific binding member, e.g., a fluorescently labelled HER-2/neu specific binding member. The standard beads and oncogene expression product-specific binding member can be present in the container in any convenient form, e.g., a storage stable composition. In certain embodiments, the standard beads and oncogene expression product-specific binding member are present in solution that maintains the integrity of these components, whereas in other embodiments the set of standard fluorescently labelled beads and the fluorescently labelled and oncogene expression product-specific binding member are present in the container in a lyophilized form.

Once the fluorescence emission for the cells in the labelled cellular sample and the standard beads are obtained, this information is used to evaluate the oncogene expression levels on the cells in the cellular sample and/or sub-populations of cells in the cellular sample.

In certain embodiments, a standard curve of emission data for the standard bead set is used to evaluate/determine the oncogene expression levels on the cells in the cellular sample. Examples of such methods can be found, for example, in a white paper describing the use of QuantiBRITE™ standard beads (Iyer S, Bishop J, Abrams, B, et al. QuantiBRITE™: A New Standard for Fluorescence Quantitation. Becton Dickinson Immunocytometry Systems, San Jose, Calif. 1997. White Paper), hereby incorporated by reference herein in its entirety. In brief, a standard curve plotting the Log₁₀ FL2 measurement versus the Log₁₀ of the known number of fluorescent labels per bead is generated using the emission date from the reference standard beads. Such a standard curve can be generated using a module present in the flow cytometry software package, or other software template, used by the flow cytometer employed in the methods or the standard curve can be generated separately by a user using the emission data and information about the standard beads.

For example, the following procedure can be used to calculate the PE molecules per cell of a population of interest:

1. On a statistics spreadsheet, enter the geometric means from the Histogram Statistics view for the standard beads (e.g., including from two to six different populations of standard beads).

2. Enter the lot-specific values for the fluorescent label molecules per bead (previously determined, e.g., provided by the supplier of the standard beads).

3. Calculate the Log₁₀ for the FL geometric means for the fluorescently labeled molecules per bead.

4. Plot a linear regression of Log₁₀ fluorescent molecules per bead against Log₁₀ fluorescence (FL), using the following equation:

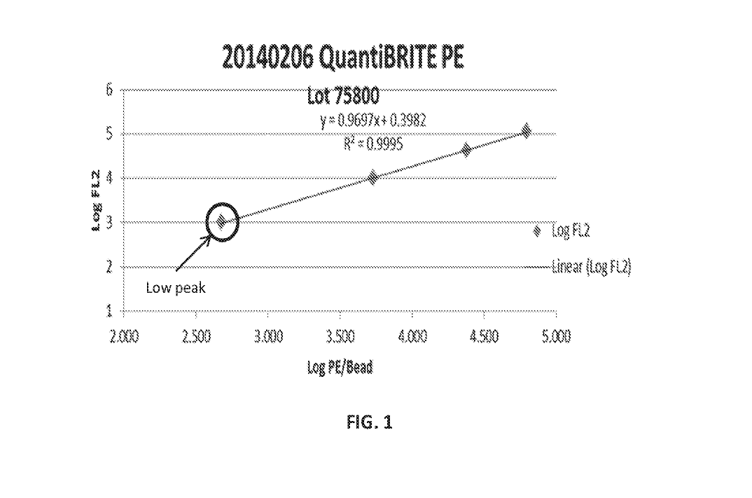

y=mx+c

where y equals Log₁₀ fluorescence and x equals Log₁₀ fluorescent molecules per bead. (An example of a standard curve of QuantiBRITE™ PE beads is shown in FIG. 1 for Lot number 75800. The diamonds represent the Log₁₀ FL2 for the four QuantiBRITE™ PE bead standards. The line represents the calculated linear regression.)

5. To determine fluorescent molecules bound per cell for an unknown cell population, substitute the Log₁₀ FL geometric means determined flow cytometrically for the unknown cells into the equation and solve for Log₁₀ fluorescent molecules per cell. Determine the anti-Log to get fluorescent molecules bound per cell.

It is noted here that fluorescent molecules bound per cell as described above is referred to in the Examples as antibodies bound per cell, or ABC, as antibodies are employed as the fluorescently labelled binding agent.

In certain embodiments, additional computational analyses are performed to evaluate the labelled cellular sample. In certain embodiments, the method includes producing a ratio-metric readout from the value of fluorescently labelled oncogene expression product-specific binding members bound per cell. For example, the fluorescently labelled oncogene expression product-specific binding members bound per cell can be divided by the number of fluorescent molecule bound per bead for on the population of beads having the lowest number of molecules (identified as the “low peak” in FIG. 1). Calculating this ratio allows a user to compare results obtained from different samples or sample runs. In other words, this ratio provides a relative comparison between different samples. This ratio can be calculated as follows:

$$Ratio = \frac{Fluorescent molecules per cell}{\begin{matrix}Fluorescent molecules per lowest \\ standard bead population\end{matrix}}$$

In addition to the ratio, a measure of the number of peaks and/or the distribution of peak width can be calculated and reported to convey the amount of variation in a clinical specimen as an indicator of tumor heterogeneity. Thus, in some embodiments, a fluorescence peak width distribution for labelled cellular sample is obtained.

In certain embodiments, the fluorescence peak width distribution is a robust coefficient of variation of intensity (rCV), which can indicate the variability within the sample. rCV can be calculated as follows (described in, e.g., Huber P J, Robust Statistics Wiley 1981 and Tech Note, Robust Statistics in BD FACSDiva Software, BD Biosciences, June 2012; hereby incorporated by reference herein in its entirety):

$$rCV = \frac{rSD}{Median FL}$$

where rSD is (Median of {|Xi−Medianx|})×1.4826 and Median FL is median fluorescence.

In certain embodiments, the expression of an additional expression product may be evaluated in the cellular sample. In such embodiments, a second fluorescently labelled expression product-specific binding member and a second set of fluorescently labelled standard beads may be employed as described above. In such embodiments, (i) the fluorescent label on the second fluorescently labelled expression product-specific binding member and the second set of fluorescently labelled standard beads is the same; and (ii) the fluorescent label on the second fluorescently labelled expression product-specific binding member and the second set of fluorescently labelled standard beads is detectably distinguishable from the fluorescent label on the first binding member and first set of fluorescently labelled beads. The identification and use of detectably distinguishable fluorescent labels in such embodiments is within the skill of the ordinary artisan. The second fluorescently labelled expression product-specific binding member can be to any desired expression product, e.g., a second oncogene or an expression product that is not an oncogene. In addition, the second expression product evaluated in the cellular sample may be expressed on the same cells (or subpopulation of cells) as the first expression product or be expressed on different cells (or different subpopulations of cells). No limitation in this regard is intended.

In certain embodiments, the expression of one or more additional expression products may be evaluated in the cellular sample, including two or more, three or more, four or more, five or more, six or more, seven or more, eight or more, nine or more, ten or more, etc. Each different expression product evaluated will be detected with a cognate binding member labelled with a detectably distinguishable fluorescent label (i.e., a label that is distinguishable from every other fluorescent label used in the assay). In addition, a set of fluorescently labelled standard beads will be included in the evaluation corresponding to each different fluorescent label used.

### Devices and Compositions

As described in detail above, the methods of the present disclosure employ flow cytometric devices to obtain emission data for a fluorescently labelled cellular sample (a cellular sample contacted with a fluorescently labelled oncogene expression product-specific binding member) and a standard set of fluorescently labelled beads to evaluate the cells in the cellular sample for oncogene expression. Thus, aspects of the present disclosure include a flow cytometer for performing the methods of the present disclosure. In certain embodiments, the flow cytometer includes (a) a flow channel comprising a labelled cellular sample containing:

(i) cells;

(ii) a fluorescently labelled oncogene expression product-specific binding member; and

(iii) a set of standard fluorescently labelled beads labelled with the same fluorescent label as the fluorescently labelled oncogene expression product-specific binding member;

(b) a light source configured to direct light to an assay region of the flow channel; and (c) a detector module configured to receive light from an assay region of the flow channel.

In certain embodiments, the fluorescently labelled oncogene expression product-specific binding member is specific for HER-2/neu, where in some embodiments the fluorescently labelled HER-2/neu specific binding member comprises a fluorescently labelled antibody, e.g., as described above.

In addition, in certain embodiments, the set of standard fluorescently labelled beads includes two or more distinct populations of beads, each having a different known amount of a conjugated fluorescent label, e.g., a “low amount” and a “high amount”, e.g., as described above.

Flow cytometer systems of interest include flow cell nozzles and optics subsystems for detecting light emitted by a sample in a flow stream. Suitable flow cytometer systems and methods for analyzing samples include, but are not limited to those described in Ormerod (ed.), Flow Cytometry: A Practical Approach, Oxford Univ. Press (1997); Jaroszeski et al. (eds.), Flow Cytometry Protocols, Methods in Molecular Biology No. 91, Humana Press (1997); Practical Flow Cytometry, 3rd ed., Wiley-Liss (1995); Virgo, et al. (2012) Ann Clin Biochem. January; 49(pt 1):17-28; Linden, et. al., Semin Throm Hemost. 2004 October; 30(5):502-11; Alison, et al. J Pathol, 2010 December; 222(4):335-344; and Herbig, et al. (2007) Crit Rev Ther Drug Carrier Syst. 24(3):203-255; the disclosures of which are incorporated herein by reference. In certain instances, flow cytometry systems of interest include BD Biosciences FACSCanto™ flow cytometer, BD Biosciences FACSVantage™, BD Biosciences FACSort™, BD Biosciences FACSCount™, BD Biosciences FACScan™, and BD Biosciences FACSCalibur™ systems, a BD Biosciences Influx™ cell sorter, BD Biosciences Jazz™ cell sorter and BD Biosciences Aria™ cell sorter, BD Biosciences FACSVerse™, BD Accuri™ C6, BD FACSCelesta™, BD LSRFortessa™, BD Biosciences FACSCanto™ II, BD LSRFortessa™ X-20, etc. or the like.

In certain embodiments, the subject systems are flow cytometer systems which incorporate one or more components of the flow cytometers described in U.S. Pat. Nos. 3,960,449; 4,347,935; 4,667,830; 4,704,891; 4,770,992; 5,030,002; 5,040,890; 5,047,321; 5,245,318; 5,317,162; 5,464,581; 5,483,469; 5,602,039; 5,620,842; 5,627,040; 5,643,796; 5,700,692; 6,372,506; 6,809,804; 6,813,017; 6,821,740; 7,129,505; 7,201,875; 7,544,326; 8,140,300; 8,233,146; 8,753,573; 8,975,595; 9,092,034; 9,095,494 and 9,097,640; the disclosures of which are herein incorporated by reference.

In addition to flow cytometric devices, aspects of the present disclosure include containers that include: (a) a fluorescently labelled oncogene expression product-specific binding member; and (b) a set of standard fluorescently labelled beads labelled with the same fluorescent label as the fluorescently labelled oncogene expression product-specific binding member, where these components are, for example, as described above.

The fluorescently labelled oncogene expression product-specific binding member and the set of standard fluorescently labelled can be present in the container in any convenient form, e.g., as a storage stable composition, including both liquid and dry, e.g., lyophilized compositions. In certain embodiments, the standard beads and oncogene expression product-specific binding member are present in a solution that maintains the integrity of these components, whereas in other embodiments the set of standard fluorescently labelled beads and the fluorescently labelled and oncogene expression product-specific binding member are present in the container in a lyophilized form.

In certain embodiments, a second fluorescently labelled expression product-specific binding member and a second set of fluorescently labelled standard beads may be present in the container. In such embodiments, (i) the fluorescent label on the second fluorescently labelled expression product-specific binding member and the second set of fluorescently labelled standard beads is the same; and (ii) the fluorescent label on the second fluorescently labelled expression product-specific binding member and the second set of fluorescently labelled standard beads is detectably distinguishable from the fluorescent label on the first binding member and first set of fluorescently labelled beads. The second fluorescently labelled expression product-specific binding member can be to any desired expression product, e.g., a second oncogene or an expression product that is not an oncogene. In certain embodiments, two or more, three or more, four or more, five or more, six or more, seven or more, eight or more, nine or more, ten or more, etc., different fluorescently labelled expression product-specific binding members and a corresponding sets of fluorescently labelled standard beads are present in the container.

Any convenient container may be used, including containers with volumetric capacities of anywhere from about 10 μl to about 100 ml, e.g., 20 μl, 50 μl, 100 μl, 200 μl, 500 μl, 1 ml, 5 ml, 10 ml, 15 ml, 25 ml, 50 ml, and 100 ml. The container can be a standard form routinely used in the relevant art, including microfuge tubes, Eppendorf tubes, conical tubes, multi-well plates, strip wells or tubes, vials, test tubes, tubes configures for flow cytometric analysis, etc. The container can be made of any desired material, e.g., plastic or glass.

### Computer Controllers and Program Products

Aspects of the present disclosure further include computer controllers and programs for operating the devices and performing calculations for evaluating oncogene expression levels on cells using fluorescence emission data from fluorescently labelled cells (e.g., contacted with a fluorescently labelled oncogene expression product-specific binding member) and a fluorescently labelled standard bead set, where the controllers further include one or more computer elements for complete automation or partial automation of a flow cytometric device as described herein.

In some embodiments, the controllers include a computer having a computer readable storage medium with a computer program stored thereon, where the computer program when loaded on the computer includes instructions for actuation of the device to perform an oncogene expression level evaluation on a cellular sample as disclosed herein, which may include any one or more of: performing a cellular staining reaction, obtaining emission data by flow cytometry of a cellular sample and a standard bead set, and employing the emission data to evaluate oncogene gene expression on the cells of the cellular sample (e.g., using the algorithms/formulas described herein).

In certain embodiments of the present disclosure, a software template for cellular oncogene expression analysis is provided. In addition, one or more algorithm to perform necessary calculations for oncogene expression product evaluation (e.g., quantitative evaluation) is provided. Such algorithms include comparison of emission data for a cellular sample to a standard curve to achieve binding members bound per cell (or antibodies bound per cell; ABC), calculation of a ratio to an internal standard, and enumeration and rCV of cellular sample peaks. Descriptions and formulas for each of these different parameters are provided above as well as in the examples below.

In certain embodiments, the controller includes an input module, a processing module and an output module. Processing modules of interest may include one or more processors that are configured and automated to implement one or more routines of the device, e.g., as described above. For example processing modules may include two or more processors, such as three or more processors, such as four or more processors and including five or more processors, configured and automated to evaluate oncogene expression of a cellular sample by obtaining emission data for the cellular sample and a corresponding standard bead set and performing the computational analyses described herein.

The controllers may include both hardware and software components, where the hardware components may take the form of one or more platforms, such that the functional elements of the controller may be carried out by the execution of software applications on and across the one or more computer platforms represented of the system.

Controllers may include a display and operator input device. Operator input devices may, for example, be a keyboard, mouse, or the like. The processing module includes a processor which has access to a memory having instructions stored thereon for performing the steps of the subject methods. The processing module may include an operating system, a graphical user interface (GUI) controller, a system memory, memory storage devices, and input-output controllers, cache memory, a data backup unit, and many other devices. The processor may be a commercially available processor or it may be one of other processors that are or will become available. The processor executes the operating system and the operating system interfaces with firmware and hardware in a well-known manner, and facilitates the processor in coordinating and executing the functions of various computer programs that may be written in a variety of programming languages, such as Java, Perl, C++, other high level or low level languages, as well as combinations thereof, as is known in the art. The operating system, typically in cooperation with the processor, coordinates and executes functions of the other components of the computer. The operating system also provides scheduling, input-output control, file and data management, memory management, and communication control and related services, all in accordance with known techniques.

The system memory may be any of a variety of known or future memory storage devices. Examples include any commonly available random access memory (RAM), magnetic medium such as a resident hard disk or tape, an optical medium such as a read and write compact disc, flash memory devices, or other memory storage device. The memory storage device may be any of a variety of known or future devices, including a compact disk drive, a tape drive, a removable hard disk drive, or a diskette drive. Such types of memory storage devices typically read from, and/or write to, a program storage medium (not shown) such as, respectively, a compact disk, magnetic tape, removable hard disk, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with the memory storage device.

In some embodiments, a computer program product is described comprising a computer usable medium having control logic (computer software program, including program code) stored therein. The control logic, when executed by the processor the computer, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Memory may be any suitable device in which the processor can store and retrieve data, such as magnetic, optical, or solid state storage devices (including magnetic or optical disks or tape or RAM, or any other suitable device, either fixed or portable). The processor may include a general purpose digital microprocessor suitably programmed from a computer readable medium carrying necessary program code. Programming can be provided remotely to processor through a communication channel, or previously saved in a computer program product such as memory or some other portable or fixed computer readable storage medium using any of those devices in connection with memory. For example, a magnetic or optical disk may carry the programming, and can be read by a disk writer/reader. Systems of the invention also include programming, e.g., in the form of computer program products, algorithms for use in practicing the methods as described above. Programming according to the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; portable flash drive; and hybrids of these categories such as magnetic/optical storage media.

The processor may also have access to a communication channel to communicate with a user at a remote location. By remote location is meant the user is not directly in contact with the system and relays input information to an input manager from an external device, such as a computer connected to a Wide Area Network (“WAN”), telephone network, satellite network, or any other suitable communication channel, including a mobile telephone (i.e., smartphone).

In some embodiments, controllers according to the present disclosure may be configured to include a communication interface. In some embodiments, the communication interface includes a receiver and/or transmitter for communicating with a network and/or another device. The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

Output controllers may include controllers for any of a variety of known display devices for presenting information to a user, whether a human or a machine, whether local or remote. If one of the display devices provides visual information, this information typically may be logically and/or physically organized as an array of picture elements. A graphical user interface (GUI) controller may include any of a variety of known or future software programs for providing graphical input and output interfaces between the system and a user, and for processing user inputs. The functional elements of the computer may communicate with each other via system bus. Some of these communications may be accomplished in alternative embodiments using network or other types of remote communications. The output manager may also provide information generated by the processing module to a user at a remote location, e.g., over the Internet, phone or satellite network, in accordance with known techniques. The presentation of data by the output manager may be implemented in accordance with a variety of known techniques. As some examples, data may include SQL, HTML or XML documents, email or other files, or data in other forms. The data may include Internet URL addresses so that a user may retrieve additional SQL, HTML, XML, or other documents or data from remote sources. The one or more platforms present in the subject systems may be any type of known computer platform or a type to be developed in the future, although they typically will be of a class of computer commonly referred to as servers. However, they may also be a main-frame computer, a work station, or other computer type. They may be connected via any known or future type of cabling or other communication system including wireless systems, either networked or otherwise. They may be co-located or they may be physically separated. Various operating systems may be employed on any of the computer platforms, possibly depending on the type and/or make of computer platform chosen. Appropriate operating systems include Windows NT®, Windows XP, Windows 7, Windows 8, iOS, Sun Solaris, Linux, OS/400, Compaq Tru64 Unix, SGI IRIX, Siemens Reliant Unix, and others.

### Kits

Also provided are kits for practicing one or more embodiments of the above-described methods. The subject kits may include various components and reagents. In some instances, the kits include at least reagents finding use in the oncogene expression evaluation methods (e.g., as described above). In addition, in certain embodiments, the kit includes a computer readable medium having a computer program stored thereon, wherein the computer program, when loaded into a computer, operates the computer to evaluate oncogene expression based on emission data obtained from cells and standard beads as described herein. In some embodiments, instead of a computer readable medium, the kit includes a physical substrate having an address from which to obtain the computer program, e.g., over the internet.

As such, certain embodiments of the disclosed kit includes (a) a fluorescently labelled oncogene expression product-specific binding member; and (b) a set of standard fluorescently labelled beads labelled with the same fluorescent label as the fluorescently labelled oncogene expression product-specific binding member, e.g., as described above.

The fluorescently labelled oncogene expression product-specific binding member and the set of standard fluorescently labelled can be present in the kit in any convenient form, e.g., as a storage stable composition in a container (as described above). In certain embodiments, the standard beads and oncogene expression product-specific binding member are present in a solution that maintains the integrity of these components, whereas in other embodiments the set of standard fluorescently labelled beads and the fluorescently labelled and oncogene expression product-specific binding member are present in a lyophilized form.

In certain embodiments, the kit includes a second fluorescently labelled expression product-specific binding member and a second set of fluorescently labelled standard beads. In such embodiments, (i) the fluorescent label on the second fluorescently labelled expression product-specific binding member and the second set of fluorescently labelled standard beads is the same; and (ii) the fluorescent label on the second fluorescently labelled expression product-specific binding member and the second set of fluorescently labelled standard beads is detectably distinguishable from the fluorescent label on the first binding member and first set of fluorescently labelled beads. The second fluorescently labelled expression product-specific binding member can be to any desired expression product, e.g., a second oncogene or an expression product that is not an oncogene. In certain embodiments, two or more, three or more, four or more, five or more, six or more, seven or more, eight or more, nine or more, ten or more, etc., different fluorescently labelled expression product-specific binding members and a corresponding sets of fluorescently labelled standard beads are present in the kit. In some of these embodiments, each combination of different fluorescently labelled expression product-specific binding member and its corresponding set of fluorescently labelled standard beads are present in a separate container in the kit, whereas in other embodiments, all of the different fluorescently labelled expression product-specific binding members and their corresponding sets of fluorescently labelled standard beads are present in the same container in the kit.

In certain embodiments, the kit can include components or reagents for obtaining and/or processing a sample from a subject to produce a cellular suspension. For example, a kit can include reagents for dissociating a tissue biopsy sample to form a cellular suspension amenable for staining and flow cytometric analysis including but not limited to: razors, needles, tissue-dissociating enzymes and/or other chemicals, cell strainers, vital dyes, and the like.

In addition to the above components, the subject kits may further include instructions for practicing the methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., CD, DVD, Blu-Ray, flash memory, etc., on which the information has been recorded. Yet another means that may be present is a website address which may be used via the Internet to access the information at a removed site. Any convenient means may be present in the kits.

### Utility

The present disclosure provides methods, compositions and kits to obtain an automated, reproducible, and quantitative value (e.g., exact value) for oncogene expression levels (e.g., for Her-2/neu) from tumor tissue (e.g., breast cancer tissue). The readout of binding members (or antibodies) bound per cell (ABC) can be reported as a ratio to the lowest internal standard bead population (e.g., from the QuantiBRITE™ kit) to provide a relative comparison between different samples. The number of peaks and distribution of peak width of a cellular sample (rCV) can be reported to convey the amount of variation in a clinical specimen as an indicator of tumor heterogeneity, which is not possible with current methods, including immunohistochemistry (IHC) and fluorescence in-situ hybridization assay, both of which are considered to have issues of accuracy, reproducibility and subjectivity.

For example, HER-2 is an actionable drug target in the treatment of breast cancer (and possibly other cancer types) and as such patient diagnosis and therapy will benefit from a reproducible and objective method for measurement of the expression of this oncogene as disclosed herein. In addition, progression of disease and resistance to therapy are thought to result from the emergence of tumor subpopulations as a response to innate microenvironment influences or therapies, and thus the ability to identify and quantitate the presence of existing subpopulations according to the present disclosure will provide valuable information for tumor characterization.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1: Standard Curve

A method to quantitatively assess and report HER-2/neu antigen expression on the surface of solid tumor cells in suspension for the purpose of subpopulation identification and quantification is performed as follows. The method quantitatively measures HER-2 antigen using flow cytometry in parallel with an internal standard (e.g., BD QuantiBRITE™ PE stained beads). BD QuantiBRITE™ beads are a fluorescence quantitation system to estimate antibodies bound per cell based on a standard curve prepared from reference beads labeled with known fluorochrome standard. Generating a standard curve involves measuring the geometric mean fluorescence (Mean FL) test wells and calculating the Log FL2.

The following procedure can be used to calculate the PE molecules per cell of the population of interest (some systems include analysis software that generate standard curves and/or calculate PE molecules per cell using QuantiBRITE™ beads).

1. On a statistics spreadsheet, enter the geometric means from the Histogram Statistics view for the four beads.

2. Enter the lot-specific values for the PE molecules per bead (provided in each QuantiBRITE™ PE kit box).

3. Calculate the Log 10 for the FL2 geometric means for the PE molecules per bead.

4. Plot a linear regression of Log 10 PE molecules per bead against Log 10 fluorescence, using the following equation:

y=mx+c

where y equals Log 10 fluorescence and x equals Log 10 PE molecules per bead. An example of a standard curve of QuantiBRITE™ PE beads is shown in FIG. 1 for Lot number 75800. The diamonds represent the Log 10 FL2 for the four QuantiBRITE™ PE bead standards. The line represents the calculated linear regression.

5. To determine antibodies bound per cell (ABC) for an unknown cell population, substitute Log FL2 geometric means in the equation and solve for Log ABC. Determine the anti-Log to get ABC.

These factors, and others that affect quantitation, are discussed in detail in the QuantiBRITE™ white paper (Iyer S, Bishop J, Abrams, B, et al. QuantiBRITE™: A New Standard for Fluorescence Quantitation. Becton Dickinson Immunocytometry Systems, San Jose, Calif. 1997. White Paper).

### Example 2: Flow Cytometric Analysis of HER-2/Neu Expression

In this Example, HER-2/neu antigen expression on individual cells in a cell suspension prepared from a solid tumor is quantified and expressed as the number of antibody molecules bound per cell (ABC).

The geometric mean of the fluorescent signal for the cell population is converted to a log FL which, using the standard curve generated using the QuantaBRITE beads, is converted to a log PE/cell which can be converted to ABC (as describe above). (It is noted here that binding agents for HER-2/neu other than antibodies may be used: as such, the term ABC is not meant to limit the use of such alternative HER-2/neu binding agents). This ABC measurement may be transformed into a ratio-metric readout by dividing the measured sample ABC by the measured value for the low peak of the standard curve (see FIG. 1, low peak of standard is circled). Thus, the formula for this ratio is:

$$Ratio = \frac{ABC}{QuantaBRITE Bead “ Low ” molecules per bead}$$

The reported measurement may also include fluorescence peak width distribution, e.g., a robust coefficient of variation of intensity (rCV), to indicate the variability within the sample. rCV can be calculated as follows (described in, e.g., Huber P J, Robust Statistics Wiley 1981 and Tech Note, Robust Statistics in BD FACSDiva Software, BD Biosciences, June 2012):

$$rCV = \frac{rSD}{Median FL}$$

where rSD is (Median of {|Xi−Medianx|})×1.4826 and Median FL is median fluorescence.

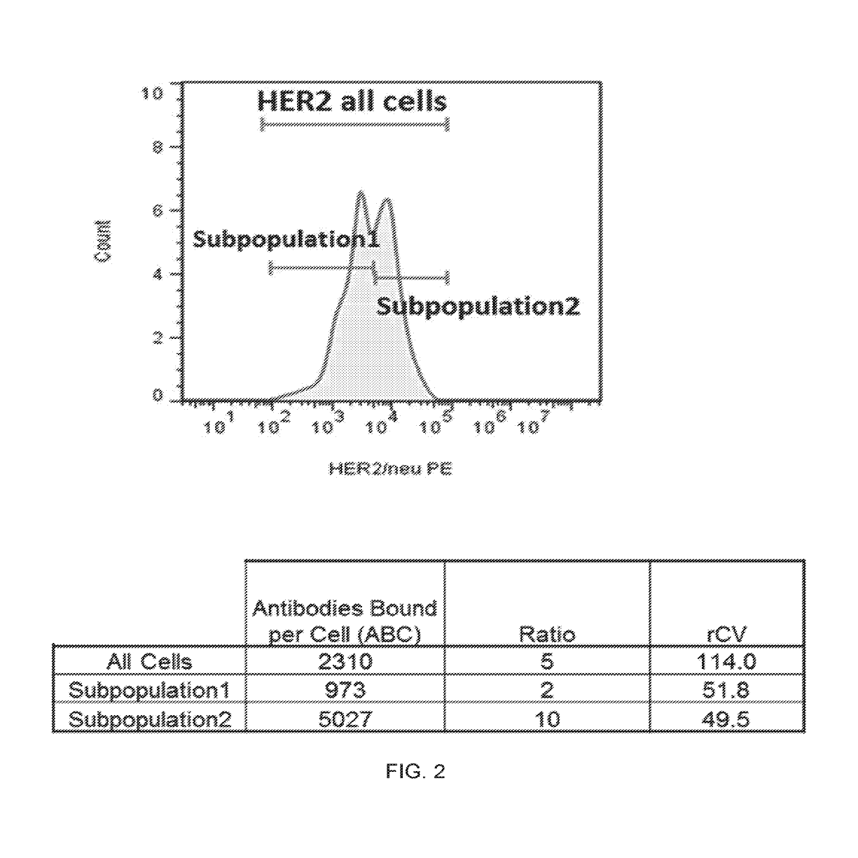

FIG. 2 shows a histogram of HER-2/neu antibody binding of the cells (top panel), with two subpopulations identified (Subpopulation 1 and Subpopulation 2). The relatively high rCV of All Cells (the high variability) allows subpopulations within the sample (1 and 2) to be identified, individually measured, and quantitated for HER-2/neu expression. The table in FIG. 2 provides the ABC, Ratio, and rCV for All Cells and Subpopulations 1 and 2, each having different levels of HER-2/neu expression. The quantitative measurement of HER-2/neu protein by flow cytometry is an improvement over current methods by combining multiple measured readouts per sample.

### Example 3: Analyses of Dynamic Range of Her-2/Neu Cultured Cell Lines and Dissociated Breast Cancer Solid Tumors from Mice

For the measurement of HER-2/neu on tumor cells, experiments were done using cultured cell lines from ATCC run on a BD Accuri C6 flow cytometer. The cell lines tested included 3 breast cancer cell lines (MCF7, MDA MB321, and SKBR3) and 3 JAX Patient Derived Xenograft (PDX) tumor models (BR0851, BR1367, and BR0291).

Staining of the PDX tumors was done by recovering the tumors directly from mice and dissociating the tumors following standard procedures (combining enzymatic and mechanical dissociation). The dissociated cells (in suspension) were stained for flow cytometry using an anti-mouse cocktail of H2Kd FITC and CD45 FITC to exclude mouse cells in parallel with PE mouse anti-Human HER-2/neu antibody (clone 24.7). The cell lines were stained with the PE mouse anti-Human HER-2/neu antibody (clone 24.7) using standard protocols (similar to the staining methods described above).

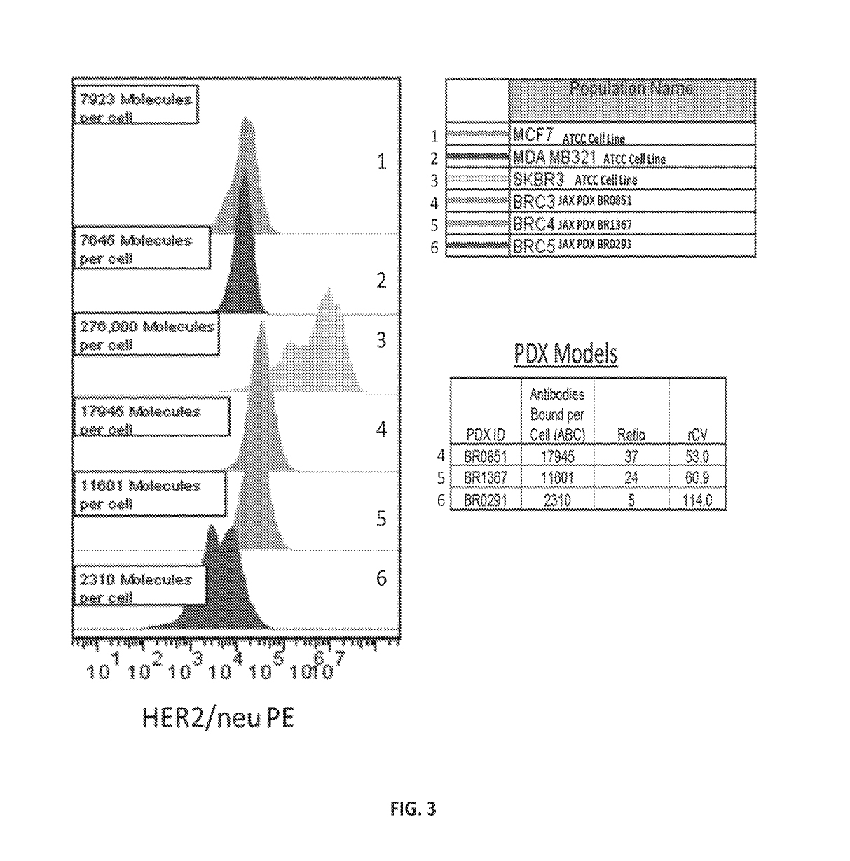

Two breast cancer cell lines that are considered negative/weakly expressive for HER-2/neu (MCF7 and MDA MB321; cell lines 1 and 2, respectively, in FIG. 3) and one cell line that is strongly positive for HER-2/neu expression (SKBR3; cell line 3 in FIG. 3) were used to develop the dynamic range for high and low marker expression. The two negative/low expressing cell lines are quantitatively similar at ˜7700 PE molecules per cell (or ABC; see the top 2 histograms, 1 and 2, in the left panel of FIG. 3) with a ratio of 16× the value of the low bead peak (ratio not listed in the table in FIG. 3). The positive cell line 3 (SKBR3) was measured at 276,000 molecules per cell (ABC) with a ratio of 564× the value of the low bead peak (ratio not shown in the table in FIG. 3).

The three JAX PDX models (BR0851, BR1367, and BR0291 in FIG. 3) show a range of expression of the HER-2/neu surface protein as seen in histograms 4, 5, and 6 in the left panel. The table in FIG. 3 shows the ABC (for HER-2/neu), Ratio, and rCV for each of these three model cells.

Further experiments were done to test an additional three PDX breast tumor models (BR0744F, BR0901, BR0705). The HER-2/neu expression (ABC) from all tumors of the PDX models tested (Jax designation BR0851, BR1367, BR0291, BR0744F, BR0901, BR0705) ranged from ˜2000 ABC for very low/negative HER-2/neu expressing tumor models (e.g., BR0291 in FIG. 3) to 95,000 ABC for high HER-2/neu expressing tumor models (e.g., BR0705, data not shown).

### Example 4: Correlation of HER-2/Neu Analysis with Gene Expression Measurement

To further support this technique as an improvement over existing methods, we performed experiments that correlate ABC measurements to gene expression measurements from PDX tumor models.

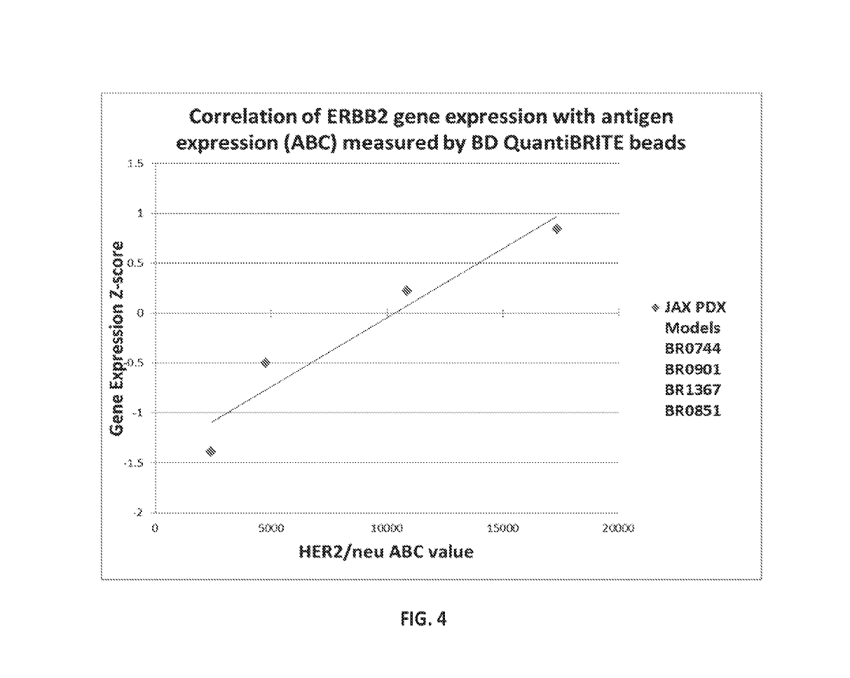

Tumors from four different PDX tumor models (BR0744, BR0901, BR1367 and BR0851) were dissociated and stained for HER-2/neu expression and ABC values calculated as described above. The calculated ABC values were plotted against the relative gene expression values (mRNA) from a reference data set from Jackson Labs for these PDX models (see FIG. 4; x-axis is ABC value and Y-axis is gene expression Z-score). The four tumor models showed agreement between total quantitative HER-2/neu expression levels (ABC) and gene expression data for HER-2/neu. This establishes a correlation between measured surface marker protein expression and gene expression in these multiple different tumor models. (Flow cytometric data for BR1367 and BR0851 is shown in FIG. 3).

### Example 5: Analysis of Six Human Breast Cancer Samples for HER-2/Neu and Comparison to Other Diagnostic Methods

Immunohistochemisry (IHC) to detect HER-2/neu protein levels and fluoresce in-situ hybriodization (FISH) to detect HER-2/neu gene copy number are techniques used clinically to score for upregulated expression of HER-2/neu in tumor section samples.

IHC is the most commonly used test to see if a tumor has too much of the HER-2/neu receptor protein on the surface of the cancer cells. The IHC test gives a score of 0 to 3+ that measures the amount of HER-2/neu receptor protein on the surface of cells in a breast cancer tissue sample. If the score is 0 to 1+, it's called “HER-2 negative.” If the score is 2+, it's called “borderline.” A score of 3+ is called “HER-2 positive.” A positive score is defined as circumferential membrane staining that is complete and intense and 1) observed in a homogenous and contiguous population and within >10% of the invasive tumor cells; and 2) readily appreciated using a low-power objective.

FISH testing is done on breast cancer tissue removed during biopsy to see if the cells have extra copies of the HER-2/neu gene. The more copies of the HER-2/neu gene that are present, the more HER-2/neu receptors the cells have. The FISH test results will tell you that the cancer is either “positive” or “negative” (a result sometimes reported as “zero”) for a HER-2/neu gene duplication event. FISH quantitative readout is based on fluorescent probe count ratio to an internal standard (i.e., a gene that is not duplicated). A positive FISH result is defined as having a ratio of >=2.0 (HER-2:Control) with an average HER-2/neu copy number <4.0 signal/cell in a homogenous and contiguous population and within >10% of the invasive tumor cells by counting at least 20 cells within the area.

Scoring tissue samples for HER-2/neu expression using IHC and FISH is well known in the art, and will not be further described herein.

Whole unprocessed clinical breast tumor tissue was purchased through Conversant Bio (Huntsville, Ala.) and shipped fresh for processing (Conversant samples 1-6). The tumors were dissociated following standard procedure using enzymatic and mechanical dissociation. Surface marker staining used a combination of FITC mouse anti-human EpCAM to identify tumor cells with PE mouse anti-human HER-2/neu. The data from the flow cytometric analysis was compared to a QuantiBRITE™ standard curve (as described above) and the ABC for total cells (“Total”) and cells above background staining (“above FMO”) were determined.

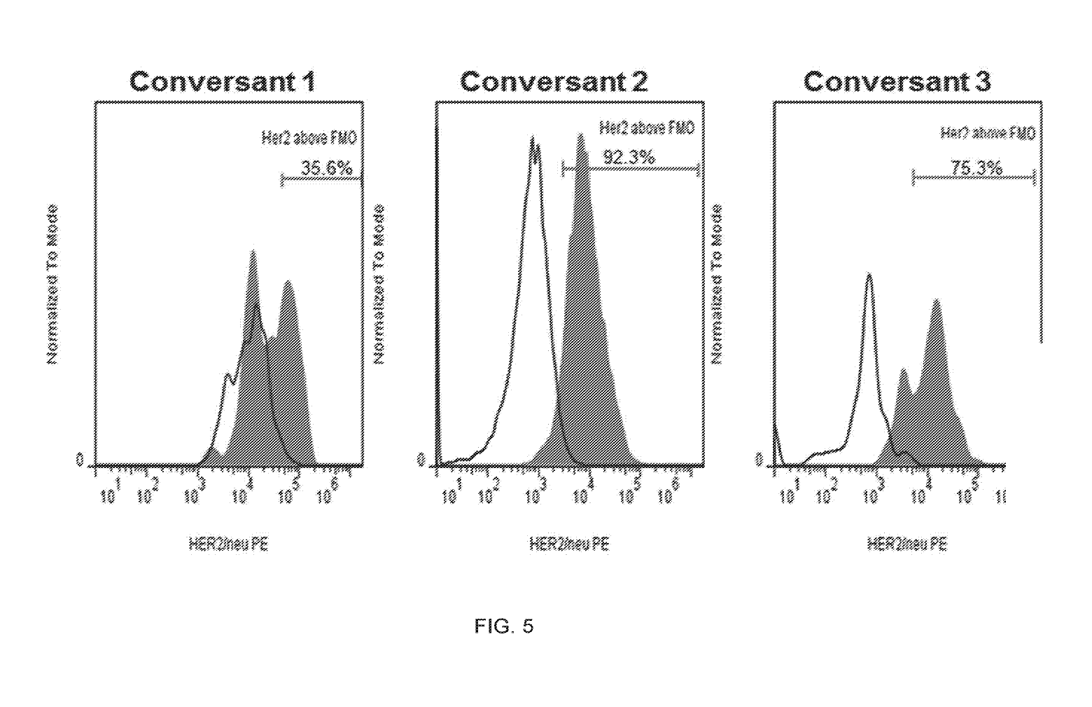

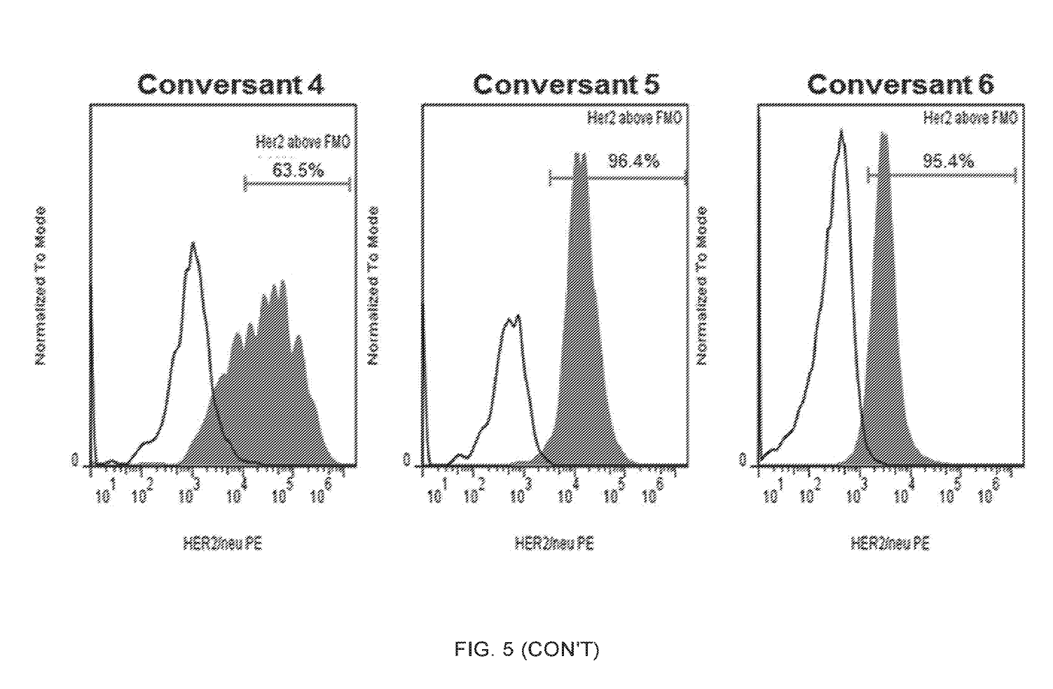

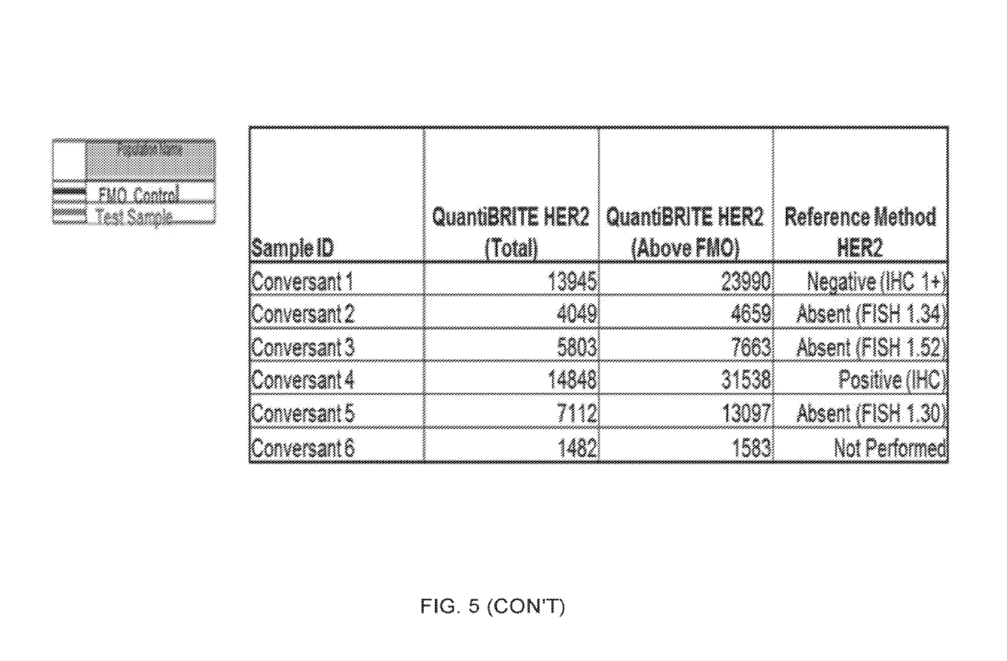

As shown in the right column of the table in FIG. 5 (“Reference Method HER-2”), all samples except Conversant 4 were either scored negative for HER-2/neu by conventional methods (IHC or FISH) or not tested (Conversant 6). Conversant 4 was scored positive for HER-2/neu expression by IHC.

Histograms for each of the six samples is shown in FIG. 5, with the negative control stained cells (line, no fill) and HER-2/new stained cells (filled histogram) shown. The table in FIG. 5 shows the calculated ABC for total cells and cells over background as well as the previously determined clinical score.

As shown in FIG. 5, Conversants 1, 2, 3, 5 and 6 (the five “negative” samples) had total HER-2/neu ABC measurements of 13945, 4049, 5803, 7112 and 1482, respectively (“Total” column in the table in FIG. 5). Conversant 4 was found to have multiple peaks with a mean measurement of 31538 ABC for cells above background staining levels (“Above FMO” column in the table in FIG. 5).

These results demonstrate the ability of the methods of the present disclosure to measure HER-2/neu over a wide range of expression levels in a cell population. The non-uniform distribution of HER-2/neu expression in many of the human breast clinical samples analyzed (see Conversants 1, 3 and 4) demonstrates the heterogeneity of expression of HER-2/neu and the ability of the flow cytometric methods disclosed herein to define subpopulations in clinical tumors based on HER-2/neu expression measured by flow. For example, the Conversant 1 human clinical breast tumor sample that was graded as negative for HER-2/neu expression by IHC (given a score of IHC 1+) displayed a bi-modal distribution of HER-2/neu expression, with a significant population of cells having increased HER-2/neu expression over background levels (see solid line background staining). Consistent with its positive IHC designation, Conversant 4 had significantly elevated HER-2/neu expression as compared to the other samples with a wide staining distribution of expression.

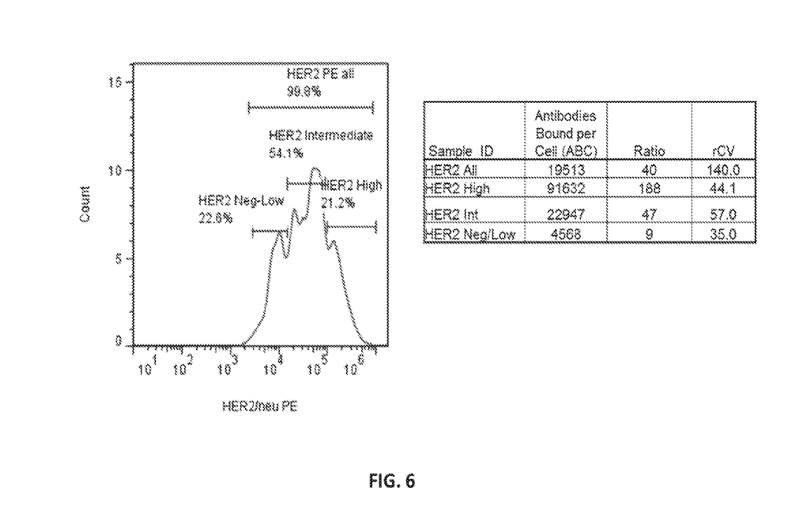

A more detailed analysis of HER-2/neu expression for Conversant 4 is shown in FIG. 6. The histogram and table in FIG. 6 demonstrate that these breast tumor tissue cells have a complex mixture of populations expressing varying levels of HER-2/neu protein. For example, a high peak comprising cells with an average ABC of 91,632 was identified as was a negative/low peak comprising cells with an average ABC of 4568 (see FIG. 6).

Using the methods provided herein, we are able to measure the percent of each subpopulation within a dissociated tumor cell sample and quantitate HER-2/neu expression and distribution within distinct populations in the sample. These methods are more sensitive and robust than classical HER-2/neu analyses (e.g., FISH and IHC). Indeed, we found that tumor model samples previously defined as negative for HER-2 expression using classical techniques did have populations of cells that expressed elevated levels of this marker. Thus, the methods disclosed herein for HER-2/neu expression profiling by flow cytometry provide a more refined diagnosis, in the form of more precise information for patient classification of HER-2/neu status, and thereby better inform the course of patient treatment.

The automated quantitative measurement of HER-2 and the mathematical expression of the sample fluorescence provide a significant improvement over currently used HER-2 assays (FISH and IHC). Furthermore, tumor subpopulations within the primary sample can be identified and quantitated based on HER-2/neu expression. The described methods provide valuable identification and measurement of discreet populations of cells for HER-2/neu measurement not possible with current methods.

Current methods for measuring HER-2/neu protein expression from a solid tumor sample are inherently lacking in the ability to provide an automated and quantitative measured value. Using flow cytometry offers a solution with an automated measure of surface marker expression and the use of BD QuantiBRITE™ beads to translate fluorescence into antibodies bound per cell (ABC). The measured fluorescence value of a stained cell suspension is translated to an exact measured value of antibodies bound per cell by measuring against a standard curve.

### EMBODIMENTS

In one embodiment, the present disclosure provides a method of evaluating a cellular sample for HER-2/neu expression, the method including flow cytometrically assaying a labelled cellular sample that has been contacted with a fluorescently labelled HER-2/neu specific binding member to obtain sample fluorescence emission data from the fluorescently labelled HER-2/neu specific binding member and employing the sample fluorescence emission data with standard fluorescence emission data obtained from a set of standard fluorescently labelled beads labelled with the same fluorescent label as the fluorescently labelled HER-2/neu specific binding member to obtain a value of fluorescently labelled HER-2/neu specific binding members bound per cell to evaluate the cellular sample for HER-2/neu expression.

In certain embodiments, the method includes producing the labelled cellular sample by contacting an initial cellular sample with the fluorescently labelled HER-2/neu specific binding member. In any above embodiments, the fluorescently labelled HER-2/neu specific binding member includes a fluorescently labelled antibody. In any above embodiments, the fluorescent label has an absorption maximum ranging from 300 to 700 nm. In any above embodiments, the fluorescent label has an emission maximum ranging from 400 to 800 nm. In any above embodiments, the fluorescent label is a phycobiliprotein. In any above embodiments, the fluorescent label is a phycoerythrin.

In any above embodiments, the set of standard fluorescently labelled beads includes two or more distinct populations of beads each having a different known amount conjugated fluorescent label, where in certain of these embodiments the set of standard fluorescently labelled beads includes two to six distinct populations of beads, e.g.,
four distinct populations of beads.

In any above embodiments, the method includes obtaining the standard fluorescence emission data. In certain of these embodiments, the standard fluorescence emission and sample fluorescence emission data are obtained at the same time. For example, the method can include contacting an initial cellular sample with a fluorescently labelled HER-2/neu specific binding member and a set of standard fluorescently labelled beads labelled with the same fluorescent label as the fluorescently labelled HER-2/neu specific binding member to obtain the labelled cellular sample. In certain of these embodiments, the contacting includes introducing the initial cellular sample into a container comprising the set of standard fluorescently labelled beads and the fluorescently labelled HER-2/neu specific binding member. In certain embodiments, the set of standard fluorescently labelled beads and the fluorescently labelled HER-2/neu specific binding member are lyophilized.

In any above embodiments, the initial cellular sample includes cells from a tissue biopsy. In certain embodiments, the tissue biopsy is a tumor biopsy, e.g., a breast cancer biopsy. In certain embodiments, the method further comprises producing the initial cellular sample from the tissue biopsy.

In any above embodiments, the method includes producing a ratiometric readout from the value of fluorescently labelled HER-2/neu specific binding members bound per cell. In any above embodiments, the method further comprises obtaining a fluorescence peak width distribution for labelled cellular sample.

In another embodiment, the present disclosure provides a flow cytometer that includes:

(a) a flow channel comprising a labelled cellular sample comprising:
(i) cells;
(ii) a fluorescently labelled HER-2/neu specific binding member; and
(iii) a set of standard fluorescently labelled beads labelled with the same fluorescent label as the fluorescently labelled HER-2/neu specific binding member;

(b) a light source configured to direct light to an assay region of the flow channel; and

(c) a detector module configured to receive light from an assay region of the flow channel.

In certain embodiments, the fluorescently labelled HER-2/neu specific binding member comprises a fluorescently labelled antibody. In any above embodiments, the fluorescent label has an absorption maximum ranging from 300 to 700 nm. In any above embodiments, the fluorescent label has an emission maximum ranging from 400 to 800 nm. In any of the above embodiments, the fluorescent label is a phycobiliprotein. In any above embodiments, the fluorescent label is a phycoerythrin. In any above embodiments, the set of standard fluorescently labelled beads comprises two or more distinct populations of beads each having a different known amount conjugated fluorescent label. In certain of these embodiments, the set of standard fluorescently labelled beads comprises two to six distinct populations of beads, e.g., four distinct populations of beads.

In another embodiment, the present disclosure provides a container comprising:
(a) a fluorescently labelled HER-2/neu specific binding member; and
(b) a set of standard fluorescently labelled beads labelled with the same fluorescent label as the fluorescently labelled HER-2/neu specific binding member.

In certain embodiments, the fluorescently labelled HER-2/neu specific binding member comprises a fluorescently labelled antibody. In any above embodiments, the fluorescent label has an absorption maximum ranging from 300 to 700 nm. In any above embodiments, the fluorescent label has an emission maximum ranging from 400 to 800 nm. In any above embodiments, the fluorescent label is a phycobiliprotein. In any above embodiments, the fluorescent label is a phycoerythrin. In any above embodiments, the set of standard fluorescently labelled beads comprises two or more distinct populations of beads each having a different known amount conjugated fluorescent label. In certain embodiments, the set of standard fluorescently labelled beads comprises two to six distinct populations of beads, e.g., four distinct populations of beads. In any above embodiments, the fluorescently labelled HER-2/neu specific binding member and the set of standard fluorescently labelled beads are lyophilized.

In another embodiment, the present disclosure provides a kit comprising:
(a) a fluorescently labelled HER-2/neu specific binding member; and
(b) a set of standard fluorescently labelled beads labelled with the same fluorescent label as the fluorescently labelled HER-2/neu specific binding member.

In certain embodiments, the fluorescently labelled HER-2/neu specific binding member comprises a fluorescently labelled antibody. In any above embodiments, the fluorescent label has an absorption maximum ranging from 300 to 700 nm. In any above embodiments, the fluorescent label has an emission maximum ranging from 400-8000 nm. In any of the above embodiments, the fluorescent label is a phycobiliprotein. In any above embodiments, the fluorescent label is a phycoerythrin. In any above embodiments, the set of standard fluorescently labelled beads comprises two or more distinct populations of beads each having a different known amount conjugated fluorescent label. In any above embodiments, the set of standard fluorescently labelled beads comprises two to six distinct populations of beads, e.g., four distinct populations of beads. In any above embodiments, the fluorescently labelled HER-2/neu specific binding member and the set of standard fluorescently labelled beads are lyophilized.

Notwithstanding the appended clauses, the disclosure set forth herein is also defined by the following clauses:

1. A method of evaluating a cellular sample for HER-2/neu expression, the method comprising:

flow cytometrically assaying a labelled cellular sample that has been contacted with a fluorescently labelled HER-2/neu specific binding member to obtain sample fluorescence emission data from the fluorescently labelled HER-2/neu specific binding member;

employing the sample fluorescence emission data with standard fluorescence emission data obtained from a set of standard fluorescently labelled beads labelled with the same fluorescent label as the fluorescently labelled HER-2/neu specific binding member to obtain a value of fluorescently labelled HER-2/neu specific binding members bound per cell;

to evaluate the cellular sample for HER-2/neu expression.

2. The method according to Clause 1, wherein the method comprises producing the labelled cellular sample by contacting an initial cellular sample with the fluorescently labelled HER-2/neu specific binding member.
3. The method according to Clause 1 or 2, wherein the fluorescently labelled HER-2/neu specific binding member comprises a fluorescently labelled antibody.
4. The method according to any of Clauses 1 to 3, wherein the fluorescent label has an absorption maximum ranging from 300 to 700 nm.
5. The method according to any of Clauses 1 to 4, wherein the fluorescent label has an emission maximum ranging from 400 to 800 nm.
6. The method according to any of the preceding clauses, wherein the fluorescent label is a phycobiliprotein.
7. The method according to Clause 6, wherein the fluorescent label is a phycoerythrin.
8. The method according to any of the preceding clauses, wherein the set of standard fluorescently labelled beads comprises two or more distinct populations of beads each having a different known amount conjugated fluorescent label.
9. The method according to Clause 8, wherein the set of standard fluorescently labelled beads comprises two to six distinct populations of beads.
10. The method according to Clause 9, wherein the set of standard fluorescently labelled beads comprises four distinct populations of beads.
11. The method according to any of the preceding clauses, wherein the method comprises obtaining the standard fluorescence emission data.
12. The method according to Clause 11, wherein the standard fluorescence emission and sample fluorescence emission data are obtained at the same time.
13. The method according to Clause 12, wherein the method comprises contacting an initial cellular sample with a fluorescently labelled HER-2/neu specific binding member and a set of standard fluorescently labelled beads labelled with the same fluorescent label as the fluorescently labelled HER-2/neu specific binding member to obtain the labelled cellular sample.
14. The method according to Clause 13, wherein the contacting comprises introducing the initial cellular sample into a container comprising the set of standard fluorescently labelled beads and the fluorescently labelled HER-2/neu specific binding member.
15. The method according to Clause 14, wherein the set of standard fluorescently labelled beads and the fluorescently labelled HER-2/neu specific binding member are lyophilized.
16. The method according to any of the preceding clauses, wherein the initial cellular sample comprises cells from a tissue biopsy.
17. The method according to Clause 16, wherein the tissue biopsy is a tumor biopsy.
18. The method according to Clause 17, wherein the tumor biopsy is a breast cancer biopsy.
19. The method according to any of Clauses 16 to 18, wherein the method further comprises producing the initial cellular sample from the tissue biopsy.
20. The method according to any of the preceding clauses, wherein the method further comprises producing a ratiometric readout from the value of fluorescently labelled HER-2/neu specific binding members bound per cell.
21. The method according to any of the preceding clauses, wherein the method further comprises obtaining a fluorescence peak width distribution for labelled cellular sample.
22. A flow cytometer comprising:

(a) a flow channel comprising a labelled cellular sample comprising:
(i) cells;
(ii) a fluorescently labelled HER-2/neu specific binding member; and
(iii) a set of standard fluorescently labelled beads labelled with the same fluorescent label as the fluorescently labelled HER-2/neu specific binding member;

(b) a light source configured to direct light to an assay region of the flow channel; and

(c) a detector module configured to receive light from an assay region of the flow channel.

23. The flow cytometer according to Clause 22, wherein the fluorescently labelled HER-2/neu specific binding member comprises a fluorescently labelled antibody.
24. The flow cytometer according to Clause 22 or 23, wherein the fluorescent label has an absorption maximum ranging from 300 to 700 nm.
25. The flow cytometer according to any of Clauses 22 to 24, wherein the fluorescent label has an emission maximum ranging from 400 to 800 nm.
26. The flow cytometer according to any of Clauses 22 to 25, wherein the fluorescent label is a phycobiliprotein.
27. The flow cytometer according to Clause 26, wherein the fluorescent label is a phycoerythrin.
28. The flow cytometer according to any of Clauses 22 to 27, wherein the set of standard fluorescently labelled beads comprises two or more distinct populations of beads each having a different known amount conjugated fluorescent label.
29. The flow cytometer according to Clause 28, wherein the set of standard fluorescently labelled beads comprises two to six distinct populations of beads.
30. The flow cytometer according to Clause 29, wherein the set of standard fluorescently labelled beads comprises four distinct populations of beads.
31. A container comprising:
(a) a fluorescently labelled HER-2/neu specific binding member; and
(b) a set of standard fluorescently labelled beads labelled with the same fluorescent label as the fluorescently labelled HER-2/neu specific binding member.
   32. The container according to Clause 31, wherein the fluorescently labelled HER-2/neu specific binding member comprises a fluorescently labelled antibody.
   33. The container according to Clause 31 or 32, wherein the fluorescent label has an absorption maximum ranging from 300 to 700 nm.
   34. The container according to any of Clauses 31 to 33, wherein the fluorescent label has an emission maximum ranging from 400 to 800 nm.
   35. The container according to any of Clauses 31 to 34, wherein the fluorescent label is a phycobiliprotein.
   36. The container according to Clause 35, wherein the fluorescent label is a phycoerythrin.
   37. The container according to any of Clauses 31 to 36, wherein the set of standard fluorescently labelled beads comprises two or more distinct populations of beads each having a different known amount conjugated fluorescent label.
   38. The container according to Clause 37, wherein the set of standard fluorescently labelled beads comprises two to six distinct populations of beads.
   39. The container according to Clause 38, wherein the set of standard fluorescently labelled beads comprises four distinct populations of beads.
   40. The container according to any of Clauses 31 to 39, wherein the fluorescently labelled HER-2/neu specific binding member and the set of standard fluorescently labelled beads are lyophilized.
   41. A kit comprising:
(a) a fluorescently labelled HER-2/neu specific binding member; and
(b) a set of standard fluorescently labelled beads labelled with the same fluorescent label as the fluorescently labelled HER-2/neu specific binding member.
   42. The kit according to Clause 41, wherein the fluorescently labelled HER-2/neu specific binding member comprises a fluorescently labelled antibody.
   43. The kit according to Clause 41 or 42, wherein the fluorescent label has an absorption maximum ranging from 300 to 700 nm.
   44. The kit according to any of Clauses 41 to 43, wherein the fluorescent label has an emission maximum ranging from 400 to 800 nm.
   45. The kit according to any of Clauses 41 to 44, wherein the fluorescent label is a phycobiliprotein.
   46. The kit according to Clause 45, wherein the fluorescent label is a phycoerythrin.
   47. The kit according to any of Clauses 41 to 46, wherein the set of standard fluorescently labelled beads comprises two or more distinct populations of beads each having a different known amount conjugated fluorescent label.
   48. The kit according to Clause 47, wherein the set of standard fluorescently labelled beads comprises two to six distinct populations of beads.
   49. The kit according to Clause 47, wherein the set of standard fluorescently labelled beads comprises four distinct populations of beads.
   50. The kit according to any of Clauses 41 to 49, wherein the fluorescently labelled HER-2/neu specific binding member and the set of standard fluorescently labelled beads are lyophilized.

Although the foregoing invention has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of evaluating a cellular sample for HER-2/neu expression, the method comprising:
flow cytometrically assaying a labelled cellular sample that has been contacted with a fluorescently labelled HER-2/neu specific binding member to obtain sample fluorescence emission data from the fluorescently labelled HER-2/neu specific binding member;
employing the sample fluorescence emission data with standard fluorescence emission data obtained from a set of standard fluorescently labelled beads labelled with the same fluorescent label as the fluorescently labelled HER-2/neu specific binding member to obtain a value of fluorescently labelled HER-2/neu specific binding members bound per cell;
to evaluate the cellular sample for HER-2/neu expression.

2. The method according to claim 1, wherein the method comprises producing the labelled cellular sample by contacting an initial cellular sample with the fluorescently labelled HER-2/neu specific binding member.

3. The method according to claim 1, wherein the fluorescently labelled HER-2/neu specific binding member comprises a fluorescently labelled antibody.

4. The method according to claim 1, wherein the set of standard fluorescently labelled beads comprises two or more distinct populations of beads each having a different known amount conjugated fluorescent label.

5. The method according to claim 1, wherein the method comprises obtaining the standard fluorescence emission data.

6. The method according to claim 1, wherein the initial cellular sample comprises cells from a tissue biopsy.

7. The method according to claim 6, wherein the tissue biopsy is a tumor biopsy.

8. The method according to claim 7, wherein the tumor biopsy is a breast cancer biopsy.

9. The method according to claim 1, wherein the method further comprises producing a ratiometric readout from the value of fluorescently labelled HER-2/neu specific binding members bound per cell.

10. The method according to claim 1, wherein the method further comprises obtaining a fluorescence peak width distribution for labelled cellular sample.

11. A flow cytometer comprising:
(a) a flow channel comprising a labelled cellular sample comprising:
(i) cells;
(ii) a fluorescently labelled HER-2/neu specific binding member; and
(iii) a set of standard fluorescently labelled beads labelled with the same fluorescent label as the fluorescently labelled HER-2/neu specific binding member;
(b) a light source configured to direct light to an assay region of the flow channel; and
(c) a detector module configured to receive light from an assay region of the flow channel.

12. (canceled)

13. A kit comprising:
(a) a fluorescently labelled HER-2/neu specific binding member; and
(b) a set of standard fluorescently labelled beads labelled with the same fluorescent label as the fluorescently labelled HER-2/neu specific binding member.

14. The kit according to claim 13, wherein the fluorescently labelled HER-2/neu specific binding member comprises a fluorescently labelled antibody.

15. The kit according to claim 13, wherein the fluorescently labelled HER-2/neu specific binding member and the set of standard fluorescently labelled beads are lyophilized.

16. The flow cytometer according to claim 11, wherein the fluorescently labelled HER-2/neu specific binding member comprises a fluorescently labelled antibody.

17. The flow cytometer according to claim 11, wherein the fluorescent label has an absorption maximum ranging from 300 to 700 nm and an emission maximum ranging from 400 to 800 nm.

18. The flow cytometer according to claim 11, wherein the fluorescent label is a phycobiliprotein.

19. The flow cytometer according to claim 18, wherein the fluorescent label is a phycoerythrin.

20. The flow cytometer according to claim 11, wherein the set of standard fluorescently labelled beads comprises two or more distinct populations of beads each having a different known amount conjugated fluorescent label.

21. The method according to claim 1, wherein the fluorescent label has an absorption maximum ranging from 300 to 700 nm and an emission maximum ranging from 400 to 800 nm.
